(19) United States
(12) Patent Application Publication
(10) Pub. No.: US 2024/0050028 A1
(43) Pub. Date: Feb. 15, 2024

(54) SLEEP CLASSIFICATION BASED ON MACHINE-LEARNING MODELS

(71) Applicant: Otsuka Pharmaceutical Development & Commercialization, Inc., Rockville, MD (US)

(72) Inventor: Jeffrey Martin COCHRAN, JR., Elkins Park, PA (US)

(73) Assignee: Otsuka Pharmaceutical Development & Commercialization, Inc., Rockville, MD (US)

(21) Appl. No.: 18/446,973

(22) Filed: Aug. 9, 2023

Related U.S. Application Data
(60) Provisional application No. 63/397,653, filed on Aug. 12, 2022.

(51) Int. Cl. A61B 5/00; A61B 5/349; A61B 5/0245; A61B 5/11

(52) U.S. Cl. CPC A61B 5/4812; A61B 5/743; A61B 5/7267; A61B 5/349; A61B 5/0245; A61B 5/11

(57) ABSTRACT

The disclosure relates to systems and methods of generating physiological state classifications such as sleep classifications. Physiological state classification may refer to a machine-learning model's prediction of a subject's physiological state based on sensor data. In particular, the machine-learning model may generate a sleep classification that represents a prediction of a subject's sleep stage. A sleep stage may refer to whether the subject is awake or asleep (for a binary classification). In some examples, the sleep stage may refer to whether the subject is awake, N1, N2, N3, and Rapid Eye Movement (REM) (for a multi-class classification).

### CROSS-REFERENCE TO RELATED APPLICATIONS

This application claims the benefit of the U.S. Provisional Patent Application No. 63/397,653; filed Aug. 12, 2022, and entitled “SLEEP CLASSIFICATION BASED ON MACHINE-LEARNING MODELS,” which is incorporated herein by reference in its entirety.

### BACKGROUND

Determining physiological states of a subject may involve lengthy visits to a laboratory that may use a wide range of equipment. For example, to determine sleep behavior, a polysomnography (“PSG”) may be used to determine a subject's sleep stages. However, a PSG may involve complicated measurements of the body including measurements of brain waves, eye movements, blood oxygen levels, and breathing patterns, among others. Additionally, a PSG typically involves monitoring the subject during sleep at a laboratory facility.

### BRIEF DESCRIPTION OF THE DRAWINGS

Features of the present disclosure may be illustrated by way of example and not limited in the following Figure(s), in which like numerals indicate like elements, in which:

FIG. 1 illustrates an example of a system for classifying a physiological state of a subject, such as whether the subject is asleep or awake;

FIG. 2 illustrates an example of training a machine-learning model to generate physiological state classifications and in particular to sleep classifications based on features derived from sensor data;

FIG. 3 illustrates a plot of a sleep fraction histogram showing a bias toward asleep data versus awake data;

FIG. 4A illustrates an example of a sampling rate of accelerometer and ECG data in a time series with PSG labeling data;

FIG. 4B illustrates an example of down-sampled accelerometer data and maintained sampling rate of ECG data in a time series with PSG labeling data;

FIG. 4C illustrates another example of down-sampled accelerometer data and maintained sampling rate of ECG data in a time series with PSG labeling data;

FIG. 5A illustrates plots of model performance at a sampling rate of accelerometer and ECG data in a time series with PSG labeling data shown in FIG. 4A;

FIG. 5B illustrates plots showing the effects of varying the sampling rate of ACC data while maintaining the sampling rate of the ECG data;

FIG. 5C illustrates plots showing the effects of varying the sampling rate of ACC data while maintaining the sampling rate of the ECG data and also showing a plot point of reduced ECG data sampling rate;

FIG. 6A illustrates an example of a sampling rate of accelerometer and continuous ECG data in a time series with PSG labeling data;

FIG. 6B illustrates an example of a sampling rate of accelerometer data and continuous ECG data for a portion of a window in a time series with PSG labeling data;

FIG. 7A illustrates plots showing the effects of varying the sampling rate of ECG data while maintaining the sampling rate of the ACC data at fifteen ACC blocks per window;

FIG. 7B illustrates plots showing the effects of varying the sampling rate of ECG data while maintaining the sampling rate of the ACC data at fifteen ACC blocks per window overlaid with the effects of varying the sampling rate of ECG data while maintaining the sampling rate of the ACC data at five ACC blocks per window;

FIG. 8 illustrates an example of a method of generating physiological state classifications based on the machine-learning model;

FIG. 9 illustrates an example of a method of generating sleep classifications based on the machine-learning model; and

FIG. 10 illustrates an example of a computing system implemented by one or more of the features illustrated in FIG. 1.

### DETAILED DESCRIPTION

The disclosure relates to systems and methods of generating physiological state classifications such as sleep classifications. Physiological state classification may refer to a machine-learning model's prediction of a subject's physiological state based on sensor data. In particular, a machine-learning model may generate a sleep classification that represents a prediction of a subject's sleep stage. A sleep stage may include an indication of whether the subject is awake or asleep (for a binary classification). Alternatively, the sleep stage may include an indication of whether the subject is in one of multiple sleep stages such as awake, N1, N2, N3, and Rapid Eye Movement (REM) (for a multi-class classification).

The system may train the machine-learning model based on sensor data that measures the subject while the subject is also monitored by a gold standard sleep test, such as a PSG. The PSG may be a reliable way to determine when a subject is in a certain sleep stage (such as awake or asleep). During the PSG, the subject may also be measured by a sensor device that includes one or more sensors. The sensor device may be a wearable device such as a patch that is worn by the user during the PSG. The system may use a mapping function that maps features derived from sensor data of the subject to known sleep stages determined by the PSG. Thus, the system may train a machine-learning model to learn features derived from sensor data that correlate with known sleep stages.

Various issues have been recognized by the inventor in connection with training and using a machine-learning model to generate sleep classifications. These issues may include noisy data, sparse data, classification bias, uniqueness of physiological data for each subject, and power consumption of sensor devices. These and other issues may hinder systems and methods that train and use machine-learning models to generate sleep classifications.

Noisy data may result from attempting to learn from and fit features that do not correlate with target outcomes. For example, certain types of sensor data may not be predictive of a subject's sleep stage. The result of noisy data is that a machine-learning model may be trained to overfit data, causing less accurate sleep classifications. To address noise, the system may implement feature selection in which non-relevant features derived from the sensor data are filtered out of a feature set. As such, feature selection may reduce noise and overfitting.

Data sparseness is a problem generally for training and using machine-learning models. With sensor-based sleep classification in particular, there may be insufficient sensor data at a given observed window to accurately make a sleep classification. To address this sparseness issue, the system may implement adaptive sampling for sensor data that dynamically increases the amount of sensor data that is generated. Adaptive sampling may be initiated by a triggering event that indicates more sensor data would be beneficial for sleep classification (or is unneeded to conserve power).

Classification bias may occur when training data is biased toward one type of classification. For example, in a PSG, classification bias may occur because most subjects are asleep during the PSG, skewing measured data toward features that support asleep classifications and away from features that support awake classifications. Thus, the PSG may bias sensor data away from the “awake” sleep classification. To address this classification bias, the system may train a machine-learning model that retains memory of prior windows of sleep classifications and may use feature data from neighboring windows. For example, the machine-learning model may be trained on and use differences in feature values between a current window and a prior window, thereby using the differences as features for training. This may enhance the model's ability to detect changes in sleep stage between windows such as from awake to asleep or vice versa, reducing classification bias of being mostly asleep.

The uniqueness of physiological data of each subject also presents challenges to machine-learning systems. For example, one subject may have different heartrates while sleeping than another subject. To address this issue, the system may generate feature sets that normalize measured sensor data for each subject, such as by generating a z-score for a given feature.

Power consumption issues generally affect any device. In particular, a sensor device that continuously (or too frequently) operates to measure a subject may use too much power. If the sensor device runs on battery or other portable power supply, it may not be feasible to continuously operate one or more of its sensors. To address this issue, the system may implement adaptive sensor data sampling in which the sampling rate of one or more sensors of the sensor device are dynamically increased when more data is needed and decreased when unnecessary to save power. These and other solutions may improve performance in training and using a machine-learning model to generate sleep classifications based on features derived from sensor data.

The one or more sensors that generate the sensor data may include an accelerometer that measures an acceleration of the subject, an electrocardiogram (ECG) sensor that measures electrical signals from the heart of the subject to generate a heartrate, and/or other types of sensors. The one or more sensors may be housed in a sensor device that measures the subject. The sensor device may be a wearable device configured as a patch, a smartwatch, an armband, and/or other form factors. Advantageously, the sensor device may be operated by the subject outside of a laboratory or other traditional sleep study settings.

The machine-learning model may use the sensor data to predict whether the subject is asleep or awake. The machine-learning model may be trained using a feature set derived from the sensor data. The feature set may be filtered during a feature selection process that optimizes model performance to identify a filtered feature set that is used for sleep classification. The filtered feature set may be identified from sensor data from a single sensor (such as accelerometer data alone or ECG data alone) or from multiple sensors (such as both accelerometer data and ECG data, or other combinations of sensor data).

After training the machine-learning model, the system may operate to generate a sleep classification based on windows of sensor data in a time series of sensor data. A window of sensor data (or simply, “window”) may refer to an interval of time during which sensor data that is received or generated is used to make a sleep classification. For example, the window may be five minutes, although other intervals of time may be used. Generally, though not necessarily, the windows may be regularly interspersed throughout the time series of sensor data. For example, continuing the five-minute window example, the machine-learning model may generate a sleep classification for every five minute window. Within each window, the system may generate feature values for the filtered feature set based on the sensor data in the window. In particular, using the values for the filtered feature set, the system may generate a feature vector suitable for input to the machine-learning model.

In some examples, the sensor data received during the time series of data may be generated or received at a certain sampling rate. For example, accelerometer data may be generated or received at a sampling rate of three times per minute, or fifteen times per five-minute window. Other sampling rates may be used, including a continuous sampling rate, which refers to generating or receiving the sensor data without pausing. It should be noted that different sensors may have the same or different sampling rates. For example, the accelerometer data may be generated or received at a first sampling rate and the ECG data may be generated or received at a second sampling rate in which the first sampling rate and the second sampling rate are the same or different from one another.

In some examples, the system may perform adaptive sampling in which the sampling rate is dynamically adjusted. For example, the sampling rate of sensor data from a given sensor may be increased (up-sampled). Up-sampling will increase the amount of sensor data available to the machine-learning model for enhanced classification resolution. Up-sampling may include increasing from a first non-continuous sampling rate to a higher second non-continuous sampling rate or from a non-continuous sampling rate to a continuous sampling rate. In another example, the sampling rate of sensor data from the given sensor may be decreased (down-sampled). Down-sampling may decrease energy consumption of the sensor and any transmitter that transmits the sensor data, which is advantageous when enhanced classification resolution is unnecessary. Down-sampling may include decreasing from a first non-continuous sampling rate to a lower second non-continuous sampling rate or from a continuous sampling rate to a non-continuous sampling rate.

In some examples, adaptive sampling may be initiated by a triggering event. The triggering event may indicate that more sensor data should be collected, which may trigger up-sampling. Alternatively, the triggering event may indicate that less sensor data is sufficient, which may trigger down-sampling. In some examples, the sensor data from a first sensor may be a triggering event for a second sensor. For example, accelerometer data may indicate reduced levels of activity of the subject during a given window or set of windows, suggesting that the subject is asleep or about to fall asleep. Such reduced levels may be a triggering event to up-sample the ECG data, causing the ECG sensor to increase the sampling rate of the ECG data. Increased ECG data during a subsequent window may be able to generate better sleep classifications. More generally, one example of a triggering event may be a predicted change in the sleep classification that will occur such as from one window to a subsequent window. The system may aggregate the sleep classifications of the observed windows and generate a sleep profile that indicates whether and when the subject has been asleep throughout the observed windows. The system may use the sleep profile to determine an amount of time that the subject slept during a given monitoring period such as overnight. The system may be used to monitor sleep of healthy individuals to gauge sleep patterns but also to monitor unhealthy individuals in which sleep is correlated with illness. Such sleep monitoring may be used to assess treatment efficacy. For example, the system may be used to monitor sleep of subjects diagnosed with severe mental illness (SMI). SMI may refer to a condition in which a subject is diagnosed with at least one mental disorder that lasts for 12-months and leads to substantial life interference. SMI is highly correlated with sleep disruption. As such, the system may be used to monitor efficacy of the treatment of SMI by monitoring quality of sleep for subjects having SMI. Subjects with milder conditions such as insomnia may be monitored as well to assess treatment efficacy.

Having described a high level overview of various features of the disclosure, attention will now turn to an example of a system for generating a physiological state classification, and more particularly a sleep classification of a subject.

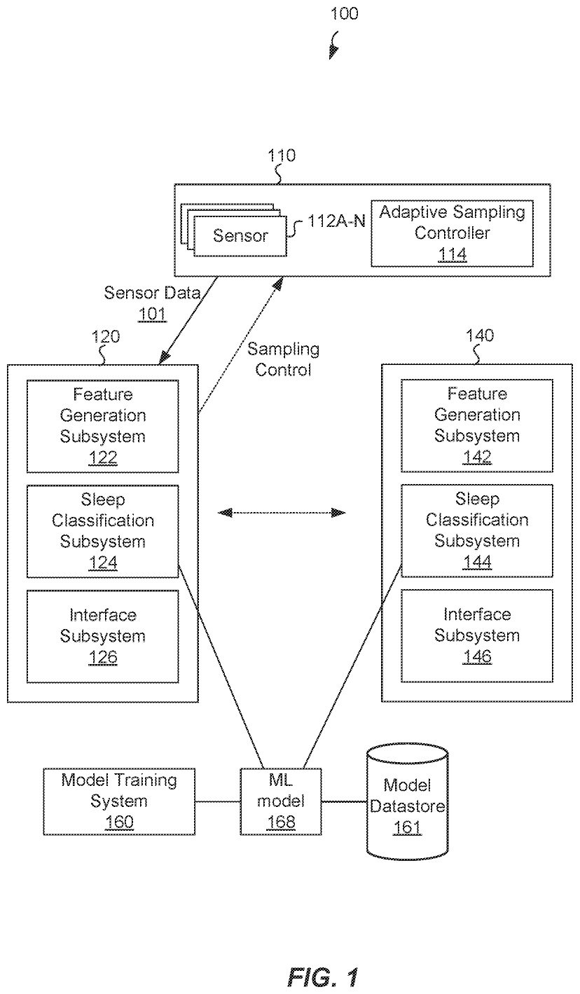

For example, FIG. 1 illustrates an example of a system 100 for classifying a physiological state of a subject, such as whether the subject is asleep or awake. System 100 may include a sensor device 110, a computing device 120, a computing device 140, a model training system 160, and/or other features. The various components of the system 100 may be connected to one another via one or more networks. One or more of the networks may be a communications network including one or more Internet Service Providers (ISPs). Each ISP may be operable to provide Internet services, telephonic services, and the like, to one or more devices, such as the computing device 120 and computing device 140. In some examples, one or more of the networks may facilitate communications via one or more communication protocols, such as those mentioned above (for example, TCP/IP, HTTP, WebRTC, SIP, WAP, Wi-Fi (for example, 802.11 protocol), Bluetooth, radio frequency systems (for example, 900 MHz, 1.4 GHz, and 5.6 GHz communication systems), cellular networks (for example, GSM, AMPS, GPRS, CDMA, EV-DO, EDGE, 3GSM, DECT, IS 136/TDMA, iDen, LTE or any other suitable cellular network protocol), infrared, BitTorrent, FTP, RTP, RTSP, SSH, and/or VOIP.

The sensor device 110 may include one or more sensors 112A-N, an adaptive sampling controller 114, and/or other components. Each sensor 112 may measure a respective aspect of a subject. For example, a sensor 112A may be an accelerometer and a sensor 112B may be an ECG sensor. Other sensors 112N may be used as well or instead. In some examples, the sensor device 110 may be configured as a wearable device. The term “wearable” may refer to being attachable to the subject via an attachment mechanism. The attachment mechanism may include a chemical mechanism such as an adhesive, a mechanical mechanism such as a strap, and/or other types of mechanisms that can attach the sensor device to a body of the subject. One example of a sensor device 110 configured as a wearable device is described U.S. Pat. No. 9,681,842, entitled “Pharma-informatics system,” issued Jun. 20, 2017, which is incorporated by reference in its entirety herein for all purposes.

The adaptive sampling controller 114 may be implemented as firmware (or other instructions) or hardware that performs adaptive sampling. The adaptive sampling controller 114 may control the sampling rate of sensor data. In some examples, the adaptive sampling controller 114 may transmit a sampling rate command to the appropriate sensor 112. In some examples, the adaptive sampling controller 114 may control the sampling rate based on power supply to the appropriate sensor 112 (such as supplying or not supplying power). The sampling rate command may include a signal to increase or decrease generation and/or transmission of the sensor data. The sampling rate command may specify a level of sampling rate to increase or decrease. In other examples, the sampling rate command may specify a target sampling rate to use.

The adaptive sampling controller 114 may adaptively change the sampling rate of one or more sensors 112A-N based on a triggering event. The triggering event may cause the adaptive sampling controller 114 to up-sample or down-sample the sensor data from a given sensor 112. In some examples, different triggering events may cause the sampling rate of different sensors 112 to be adaptively adjusted.

The triggering event may include an indication that more sensor data should be generated, resulting in up-sampling such as a continuous two-minute burst or other up-sampling to improve model performance. In other instances, the triggering event may include an indication that more sensor data is unnecessary or should otherwise be reduced, resulting in down-sampling to conserve power. Examples of triggering events will now be described. These triggering events may each individually trigger adaptive sampling or may be combined with one another to together trigger adaptive sampling.

One example of a triggering event may include a determination that the subject is at rest, which may trigger up-sampling to determine whether the subject is asleep. For example, accelerometer data may indicate that the subject is at rest. In particular, a triggering event may include accelerometer data that indicates that the subject has a resting body angle less than a threshold body angle value such as 30 degrees. Alternatively, or additionally, a triggering event may include accelerometer data that indicates a step count of zero for greater than a threshold period of time. A triggering event may also operate in reverse. That is, the triggering event may indicate that additional sensor data is unnecessary, such as when the subject is no longer detected as being at rest. This may occur when the resting body angle is greater than the threshold body angle value and/or when the step count is non-zero.

Although illustrated as a standalone device remote from the computing device 120 and the computing device 140, it should be noted that the sensor device 110 or one or more of its components (such as one or more sensors 112 and/or the adaptive sampling controller 114) may be integrated within the computing device 120 and/or computing device 140. For example, the computing device 120 may include the adaptive sampling controller 114, in which case the computing device 120 may transmit sampling control commands back to the sensor device 110. In examples for which the machine-learning model 168 is computationally too expensive for the computing device 120, the computing device 120 may transmit the sensor data to the computing device 140 for feature generation and model execution. In other examples, the computing device 120 may generate the feature set from the sensor data and transmit the feature set to the computing device 140 for model execution.

The computing device 120 may include a feature generation subsystem 122, a sleep classification subsystem 124, an interface subsystem 126, and/or other components. Generally, though not necessarily, the computing device 120 may be configured as a user-operated device that allows the user to monitor the user's sleep based on the sleep classifications generated by the sleep classification subsystem 124. For example, the computing device 120 may include a laptop or desktop computer, a tablet computer, a smartphone, and/or other type of computing device.

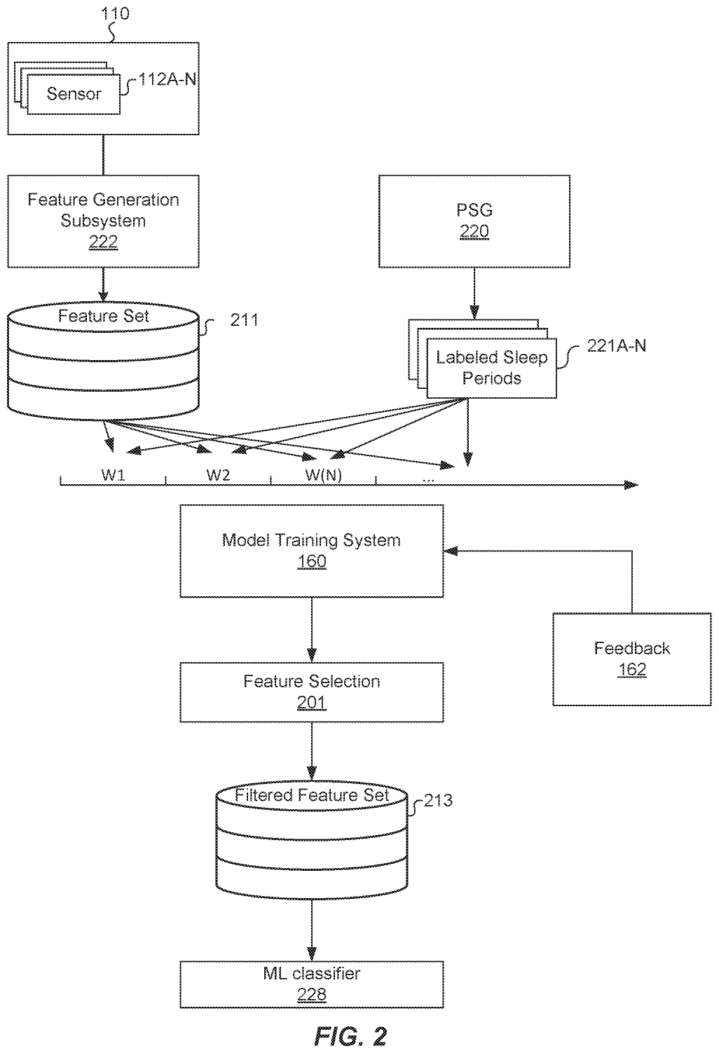

The feature generation subsystem 122 may access sensor data from the one or more sensors 112 and generate features based on the sensor data for input to the machine-learning model 168. Feature generation will be described in more detail with respect to training the machine-learning model 168 at FIG. 2. It should be noted that the feature generation subsystem 122 may generate only a filtered feature set that is input to the machine-learning model 168.

The sleep classification subsystem 124 may execute the machine-learning model 138 to generate sleep classifications. For example, the sleep classification subsystem 124 may obtain a filtered feature set generated by the feature generation subsystem 122 and provide the filtered feature set as input to the machine-learning model 138, along with the appropriate model parameters discussed with respect to FIG. 2.

The machine-learning model 168 may receive the generated features as input and generate a sleep classification based on the features. The sleep classification may include a binary classification such as either sleep or awake. In this example, the machine-learning model 168 is a binary classifier. In other examples, the sleep classification may include a single classification, such as an indication of sleep. In this example, the machine-learning model 168 is a one-class classifier. In still other examples, the sleep classification may include more than two classifications, such as an indication of wake or one or many stages of sleep.

The interface subsystem 126 may generate data for displaying the sleep classification generated by the machine-learning model 168. For example, the data may be in the form of a user interface such as a graphical user interface that is displayed on a display device. In some examples, the user interface may receive feedback data for retraining the machine-learning model 168.

The computing device 140 may include a feature generation subsystem 142, a sleep classification subsystem 144, an interface subsystem 146, and/or other components. Generally, though not necessarily, the computing device 140 may be configured as a backend computing system such as a server device that is able to determine sleep classifications. The feature generation subsystem 142, sleep classification subsystem 144, and interface subsystem 146 may operate in substantially the same manner as their counterparts in the computing device 120. The distributed functionality may permit some or all of the processing to occur at the computing device 120 and/or the computing device 140.

Training a Sleep Classifier

The model training system 160 may train a machine-learning model 168 to generate sleep classifications based on the sensor data. The model training system 160 may store the machine-learning model 168 in the model datastore 161. Storing the machine-learning model 168 may refer to storing learned parameters, model parameters used for training and/or for executing, and/or other modeling data used to generate sleep classifications on input data.

Reference will now be made to FIG. 2, which illustrates an example of training the machine-learning model 168 to generate physiological state classifications—in particular, sleep classifications—based on features derived from sensor data. The machine-learning model 168 may be trained based on an observed population of test subjects. Each test subject may be monitored by a sensor device 110. At the same time, each test subject may participate in a PSG 220, which is conducted in parallel with measurements by the sensor device 110. The PSG 220 is generally considered a gold standard test for determining sleep. Other types of sleep studies may be run in parallel with measurements by the sensor device 110. In one example training and validation, 73 test subjects were observed over 220 nights in a sleep laboratory. Of the 73 test subjects, 42 had SMI and 31 were healthy volunteers. In the sleep laboratory, a PCG was administered on each of the 73 test subjects, who also wore the sensor device 110.

Generally speaking, the sensors 112A-N of the sensor device 110 generate sensor data, which is input to the feature generation subsystem 222. It should be noted that the feature generation subsystem 122 and feature generation subsystem 142 illustrated in FIG. 1 may perform the same functions as the feature generation subsystem 222. The feature generation subsystem 222 may generate a feature set 211 based on the sensor data. The feature set 211 may include a plurality of features and their corresponding feature values that are based on the sensor data. Each feature of the feature set 211 may be associated with a corresponding window. For example, the feature set 211 may include feature values for each window for which the sensor data is generated or received.

Feature Generation (for Training and Executing)

The feature generation subsystem 222 may generate, from the sensor data, the feature set 211 illustrated in Table 1 below. In particular, feature generation subsystem 222 may generate a normalized heartrate for each specific subject by generating a z-score based on the mean and standard deviation of the subject's heartrate. This may alleviate bias introduced by subject specific heartrate that may be unique from subject to subject. In another example, the feature generation subsystem 222 may generate features that are based on neighboring windows. For example, the feature generation subsystem 222 may generate a delta, or difference, between one or more feature values across one or more previous windows and a current window. The foregoing may alleviate sparseness of data and classification bias. Further examples of features in the feature set 211 is illustrated in Table 1.

Table 1 illustrates an example of a feature set 211 derived from sensor data to train the machine-learning model 168. Each row in Table 1 is an example of a feature and its corresponding feature name, feature definition, and feature source. It should be noted that other features and sources may be used as well or instead. The feature generation subsystem 222 may generate a feature value for each of the features based on the feature definition. For example, feature generation subsystem 222 may generate a feature value for the feature “absAng_d” based on its feature definition “Absolute Value of Body Angle—Range” using accelerometer data from an accelerometer (such as a sensor 112A) to calculate a range of body angle values. In Table 1, “R-R” refers to an RR interval, which is the time elapsed between two successive R-waves of a QRS signal on the electrocardiogram and is a function of intrinsic properties of the sinus node as well as autonomic influences. In Table 1, feature names with an asterisk (*) were identified during feature selection 201 discussed below to form a filtered feature set 213. Feature names with a double asterisk (**) represent additional heart rate variability (HRV) features used in ECG sampling analysis. For the Frequency Domain HRV features shown in Table 1 (1fPeak, 1fPow, 1fPowNorm, hfPeak, hfPow, hfPowNorm, 1fhf), a periodogram (the Lomb-Scargle periodogram (LSP)) was used to generate the frequency power spectrum instead of a fast Fourier transform (FFT). The periodogram was used to overcome uneven sampling of the ECG inherent in block sampling.

| | | Feature Source |
|---|---|---|
| Feature Name | Feature Definition | (type of sensor) |
| absAng_d | Absolute Value of Body Angle - | Accelerometer |
| | Range | |
| absAng_m | Absolute Value of Body Angle - | Accelerometer |
| | Mean | |
| absAng_s | Absolute Value of Body Angle - | Accelerometer |
| | St. Dev. | |
| absXMean_d* | Absolute Value of Mean X- | Accelerometer |
| | Acceleration - Range | |
| absXMean_m* | Absolute Value of Mean X- | Accelerometer |
| | Acceleration - Mean | |
| absXMean_s | Absolute Value of Mean X- | Accelerometer |
| | Acceleration - St. Dev. | |
| absYMean_d | Absolute Value of Mean Y- | Accelerometer |
| | Acceleration - Range | |
| absYMean_m | Absolute Value of Mean Y- | Accelerometer |
| | Acceleration - Mean | |
| absYMean_s | Absolute Value of Mean Y- | Accelerometer |
| | Acceleration - St. Dev. | |
| absZMean_d | Absolute Value of Mean Z- | Accelerometer |
| | Acceleration - Range | |
| absZMean_m* | Absolute Value of Mean Z- | Accelerometer |
| | Acceleration - Mean | |
| absZMean_s | Absolute Value of Mean Z- | Accelerometer |
| | Acceleration - Standard | |
| | Deviation (St. Dev.) | |
| angle_d* | Body Angle - Range | Accelerometer |
| angle_m | Body Angle - Mean | Accelerometer |
| angle_s | Body Angle - St. Dev. | Accelerometer |
| energy_d | Energy Expenditure - Range | Accelerometer |
| energy_m | Energy Expenditure - Mean | Accelerometer |
| energy_s | Energy Expenditure - St. Dev. | Accelerometer |
| maxAxisChange | Number of Maximum | Accelerometer |
| | Acceleration Axis Changes | |
| accMean_d | Mean Total Acceleration | Accelerometer |
| | Magnitude - Range | |
| accMean_m | Mean Total Acceleration | Accelerometer |
| | Magnitude - Mean | |
| accMean_s | Mean Total Acceleration | Accelerometer |
| | Magnitude - St. Dev. | |
| xMean_d | Mean X-Acceleration - Range | Accelerometer |
| xMean_m | Mean X-Acceleration - Mean | Accelerometer |
| xMean_s | Mean X-Acceleration - St. Dev. | Accelerometer |
| yMean_d | Mean Y-Acceleration - Range | Accelerometer |
| yMean_m | Mean Y-Acceleration - Mean | Accelerometer |
| yMean_s* | Mean Y-Acceleration - St. Dev. | Accelerometer |
| zMean_d | Mean Z-Acceleration - Range | Accelerometer |
| zMean_m | Mean Z-Acceleration - Mean | Accelerometer |
| zMean_s | Mean Z-Acceleration - St. Dev. | Accelerometer |
| accStd_d | Standard Deviation of Total | Accelerometer |
| | Acceleration Magnitude - Range | |
| accStd_m | Standard Deviation of Total | Accelerometer |
| | Acceleration Magnitude - Mean | |
| accStd_s | Standard Deviation of Total | Accelerometer |
| | Acceleration Magnitude - St. | |
| | Dev. | |
| xStd_d | Standard Deviation of X- | Accelerometer |
| | Acceleration - Range | |
| xStd_m* | Standard Deviation of X- | Accelerometer |
| | Acceleration - Mean | |
| xStd_s* | Standard Deviation of X- | Accelerometer |
| | Acceleration - St. Dev. | |
| yStd_d* | Standard Deviation of Y- | Accelerometer |
| | Acceleration - Range | |
| yStd_m | Standard Deviation of Y- | Accelerometer |
| | Acceleration - Mean | |
| yStd_s | Standard Deviation of Y- | Accelerometer |
| | Acceleration - St. Dev. | |
| zStd_d | Standard Deviation of Z- | Accelerometer |
| | Acceleration - Range | |
| zStd_m* | Standard Deviation of Z- | Accelerometer |
| | Acceleration - Mean | |
| zStd_s | Standard Deviation of Z- | Accelerometer |
| | Acceleration - St. Dev. | |
| adjStepCount | Step Count | Accelerometer |
| act_d* | Total Activity - Range | Accelerometer |
| act_m* | Total Activity - Mean | Accelerometer |
| act_s | Total Activity - St. Dev. | Accelerometer |
| nZeroCross* | Number of Mean Activity | Accelerometer |
| | Crosses | |
| sdRR | Standard Deviation of R-R | ECG |
| | Values | |
| Rmssd** | Root-Mean-Square of | ECG |
| | Successive R-R Value | |
| | Difference | |
| pNN50 | Fraction of Successive R-R | ECG |
| | Differences greater than 50 | |
| | milliseconds (ms) | |
| pNN20 | Fraction of Successive R-R | ECG |
| | Differences greater than 20 ms | |
| hrvTri** | Heart Rate Variability Triangular | ECG |
| | Index | |
| hr_m | Mean Heart Rate | ECG |
| hr_med | Median Heart Rate | ECG |
| lfPeak | R-R Low-Frequency Range Peak | ECG |
| | Frequency | |
| lfPow | R-R Low-Frequency Range | ECG |
| | Power | |
| hfPeak | R-R High-Frequency Range | ECG |
| | Peak Frequency | |
| hfPow | R-R High-Frequency Range | ECG |
| | Power | |
| lfhf | Ratio of R-R Low-Frequency to | ECG |
| | High-Frequency Power | |
| IfPowNorm | Normalized R-R Low-Frequency | ECG |
| | Range Power | |
| hfPowNorm | Normalized R-R High- | ECG |
| | Frequency Range Power | |
| hr_m_z* | Mean Heart Rate Z-Score | ECG |
| hr_med_z | Median Heart Rate Z-Score | ECG |

One aspect of the disclosure relates to modeling sleep stages as a sequence. For example, the model training system 160 may model the sleep stages based on a time series of sensor data that may be classified within windows. The time series correspond to the period during which a subject is being monitored for training the machine-learning model 168. For each test subject, the PSG 220 may generate labeled sleep periods 221A-N. The labeled sleep periods 221 may indicate a time and sleep state (awake and asleep—which may include sleep stages). For example, a time interval may be labeled with a sleep label indicating whether the test subject was awake, asleep, and/or was in a specific sleep stage.

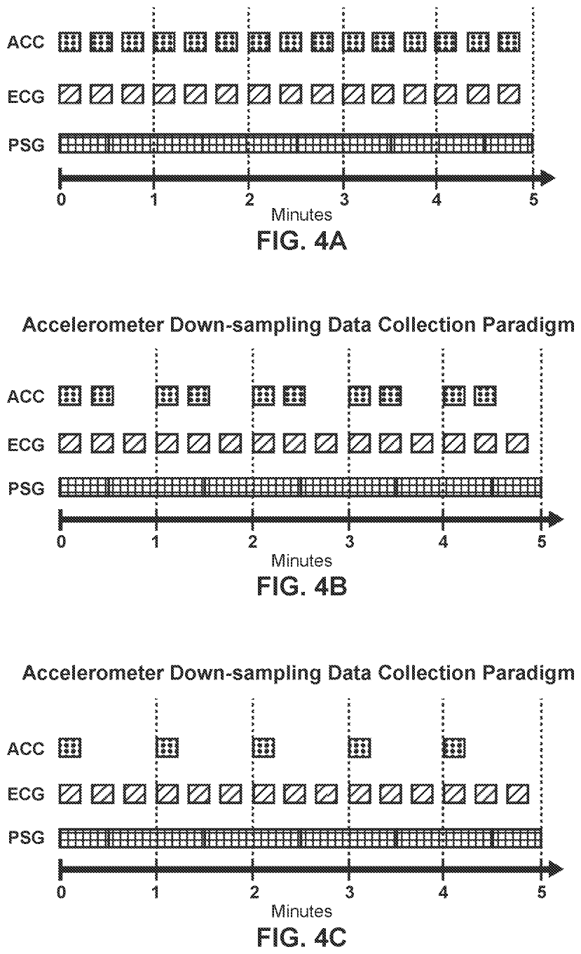

Each time interval may correspond to a respective window W1, W2, . . . , W(N) so that each window is labeled with a sleep label derived from the PSG 220 and includes sensor data generated during that window. In this way, the sensor data for a subject from the sensor device 110 may be associated with a respective sleep label for the subject as determined from the PSG 220. Examples of a window W1, W2, . . . , W(N) of sensor data for feature generation and PCG data for sleep labels are illustrated in FIGS. 4A-C and 6A-B, which each show a five-minute window.

The model training system 160 may train the machine-learning model 168 based on the feature set 211 and the labeled sleep periods 221. For example, for each window, the model training system 160 may correlate features and their feature values in the feature set 211 with corresponding sleep labels. In other words, the model training system 160 may learn correlations between a given feature from the sensor data with a sleep label. Put another way, the model training system 160 may learn a level of predictiveness each feature has on sleep stage.

The model training system 160 may train the machine-learning model 168 to generate sleep classifications according to the windows labeled with a sleep label and the features derived from sensor data for that window. Generally speaking, classification models attempt to estimate a mapping function (ƒ) from the input variables (x) to discrete or categorical output variables (y). In particular, the input variables (x) are features derived from sensor data and the output variables (y) are each a sleep classification that the mapping function predicts based on input variables (x). During training, the model training system 160 may map features derived from the sensor data (input variables (x)) to sleep labels (output variables (y)).

The model training system 160 may train the machine-learning model 168 to generate different numbers of classifications depending on the sleep labels that are generated from the PSG 220. For example, the machine-learning model 168 may be a one-class classifier, a binary classifier (which is a two-class multi-class classifier), or a multi-class classifier. A one-class classifier may generate a single classification of only asleep or only awake. For example, a one-class classifier may be trained to classify input data as being associated with being awake, but not classify asleep periods. Another one-class classifier may be trained to classify input data as being associated with being asleep, but not classify awake periods. To train the one-class classifier, the model training system 160 may use a single sleep label in the labeled sleep periods 221—either asleep or awake but not both.

A binary classifier may generate a classification of either one of asleep or awake. For example, the binary classifier may be trained to classify input data as being associated with either being awake or being asleep. To train the binary classifier, the model training system 160 may use two sleep labels in the labeled sleep periods 221—asleep and awake.

A multi-class classifier (greater than two) may generate a classification of either one of awake, N1, N2, N3, and REM. To train the multi-class classifier, the model training system 160 may use more than two labels in the labeled sleep periods 221, which may correspond to sleep stages such as awake, N1, N2, N3, and REM.

The model training system 160 may generate different types of machine-learning models 168. For example, the machine-learning model 168 may include a Conditional Random Field (CRF) model, a Long-term Short-Term Memory (LSTM) model, a Light Gradient Boosting Machine (LGBM), and/or other types of models. To illustrate, these models will be described with reference to implementation via PYTHON packages, although other implementations may be used.

The CRF Model

A CRF model is a class of discriminative modeling that makes a current prediction based on contextual information or state of neighboring predictions. In the context of sleep classification, the CRF model may incorporate sleep classifications of prior windows to make a sleep classification of a current window. For example, the CRF may look back to sleep classifications of prior N windows, where N is an integer greater 0 for the current window. If each window is five-minutes, examples of N may be 1, 2, 3, 4, or 5. Other N prior windows may be used, but N=1 was found to be more predictive than N=6 for five-minute windows. The CRF may be trained via gradient descent with limited-memory Broyden-Fletcher-Goldfarb-Shanno (1-BFGS). Such training is an iterative first-order optimization used to find a local minimum/maximum of a given function to minimize a cost/loss function. For example, the CRF may be trained iteratively learned data for the features in the feature set 211 approximate the sleep label within a predetermined threshold.

One example implementation of model training with associated model parameters may include the CRF function in the sklearn-crfsuite PYTHON package:
Generate transitions of all possible label pairs (all_possible_transitions=True)
Generate all possible state features (all_possible_states=True)
L1 Regularization coefficient (c1): 0.1
L2 Regularization coefficient (c2): 0.01
Maximum number of iterations (max iterations): 5000

For binary classifications, the transition matrix with all possible transitions from a prior window may include a 2×2 matrix: awake-to-sleep, stay awake, sleep-to-awake, and stay asleep. More complex transition matrices with all possible transitions may be similarly instantiated depending on the number of classes and therefore transitions of sleep states (such as awake-to-N1 and N1-to-N2 and so forth).

The LSTM Model

LSTM networks may overcome the long-term dependency problem faced by recurrent neural networks (RNNs) due to the vanishing gradient problem, which is caused by “vanishingly” small adjustments made to model weights that are learned during training. The result is that RNNs typically stop learning. To solve this problem, LSTMs have feedback connections that enable LSTMs to process an entire sequence of data while retaining memory about data points throughout the sequence. Thus, LSTMs provide long-term memory throughout a sequence such as a time-series of data. In this context, an LSTM may take into account all windows throughout the time series of feature data derived from the sensor data. An LSTM uses a neural network, which refers to a computational learning system that uses a network of neurons to translate a data input of one form into a desired output. A neuron may refer to an electronic processing node implemented as a computer function, such as one or more computations. The neurons of the neural network may be arranged into layers. Each neuron of a layer may receive as input a raw value, apply a classifier weight to the raw value, and generate an output via an activation function. The activation function may include a log-sigmoid function, hyperbolic tangent, Heaviside, Gaussian, SoftMax function and/or other types of activation functions.

One example implementation of model training with associated model parameters may include the keras PYTHON package, with the following LSTM layers: a recurrent LSTM (LSTM) layer, a dropout (Dropout) layer, and a dense (Dense) output layer:
Hidden layer size (units): 128
Whether to return last output (return sequences=False)
Fraction of units to drop for transformation of inputs (dropout=0.5)
Fraction of units to drop for transformation of recurrent state (recurrent dropout=0.5)
Historical number of timesteps (timesteps within Input function for past windows): 11
Future number of timesteps (timesteps within Input function for future windows): 11
Dropout parameters
Dimensionality of output space (units): 64
Activation function (activation): Rectified Linear Unit (‘relu’)
Dropout rate (rate): 0.5
Dense (output) parameters:
Dimensionality of output space (units): 1
Activation function (activation): Sigmoid (‘sigmoid’)
Initialization constant for the bias vector (bias_initializer): initializer.Constant(1.6)
Model compiled with Model.compile
Loss function (loss): Binary Cross-Entropy (‘binary_crossentropy’)
Optimizer (optimizer): Adamax (Adamax)
Learning Rate (learning_rate): 0.001
Exponential decay rate for 1st moment estimates (beta_1): 0.9
Exponential decay rate for exponentially weighted infinity norm (beta_2): 0.999
Epsilon constant (epsilon): 1e-7

The LGBM

Unlike the CRF and LSTM models, the LGBM does not use contextual information of neighboring windows. Thus, the machine-learning model 168 trained as an LGBM may use only the current window's features to make a sleep classification for the current window. Gradient boosting is a machine learning technique for regression and classification problems, which produces a prediction model in the form of an ensemble of weak prediction models, typically decision trees. LGBM may build a model in a stage-wise fashion and generalizes the model by allowing optimization of an arbitrary differentiable loss function.

One example implementation of model training with associated model parameters may include the LGBMClassifier function of the lightgbm PYTHON package and RandomizedSearchCV from scikit-learn Python package. The machine-learning model 168 is trained many different times with each iteration using a different set of hyperparameters with hyperparameters being sampled from defined distributions:
Number of times that different hyperparameters are sampled (n_iter): 1000
Number of folds for cross-validation (cv): 3
Parameter Distributions
Learning Rate (learning_rate): Uniform distribution between 0.01 and 0.49 (inclusive)
Number of Estimators (n_estimators): Random integer between 100 and 700 (inclusive)
Maximum Depth of Classifier (max_depth): Random integer between 1 a nd 10 (inclusive)
Subsample Ratio (subsample):Uniform distribution between 0.4 and 0.6 (inclusive)

Once the machine-learning model 168 is trained using any of the foregoing or other techniques, feature selection 201 may be performed to filter in or out features from the feature set 211 to generate a filtered feature set 213. The filtered feature set 213 may include a subset of the feature set 211. Feature selection 201 may refer to reducing the number of features used in a machine-learning model. The feature set may be filtered during a feature selection process that optimizes model performance to identify a filtered feature set that is used for sleep classification. Feature selection reduces noise and overfitting since different sensor data and different features derived from the sensor data may have different predictive impact on the subject's sleep stage. The filtered feature set 213 may be identified from sensor data from a single sensor (such as accelerometer data alone or ECG data alone) or from multiple sensors (such as both accelerometer data and ECG data, or other combinations of sensor data).

In some examples, the model training system 160 may access feedback 162 that indicates an actual sleep classification. For example, the subject or other user may provide an indication, such as through the interface subsystem 126, of the subject's sleep classification. For example, the subject may indicate that the machine-learning model 168 wrongly predicted that the subject was asleep during a given window. Alternatively, or additionally, a validation exercise may be conducted in which a PCG was conducted in parallel with execution of the machine-learning model 168 to further provide feedback on model performance. Such feedback may be used to retrain the machine-learning model 168.

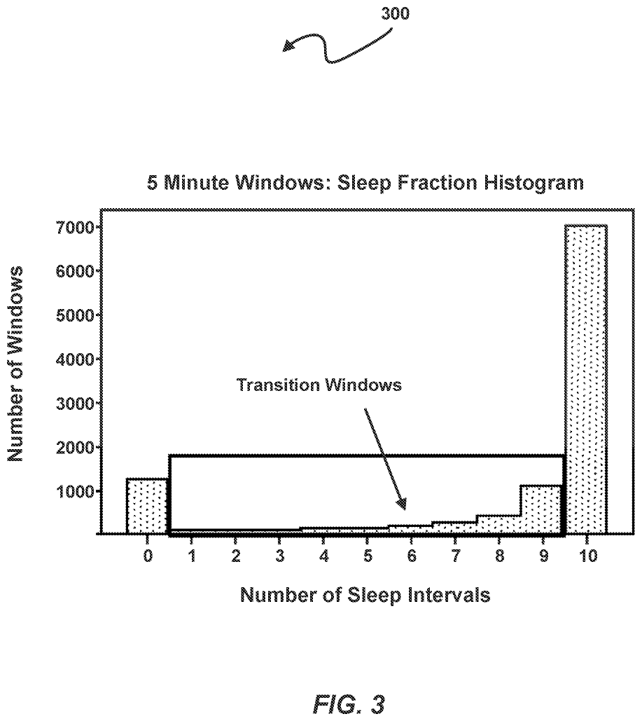

FIG. 3 illustrates a plot of a sleep fraction histogram 300 showing a bias toward asleep data versus awake data. The x-axis represents a number of sleep intervals plotted against a number of windows on the y-axis of the sleep fraction histogram 300. The sleep fraction histogram 300 shows when subjects were awake (sleep interval 0), transition between asleep and awake (sleep intervals 1-9) and asleep (sleep interval 10). Data was generated based on three ACC blocks per minute (fifteen ACC blocks per five-minute window), three ECG blocks per minute (fifteen ECG blocks per five-minute window), and continuous PSG results.

In total, the sleep fraction histogram 300 represents 10,170 five-minute windows with at least ten valid ECG/ACC blocks (5,610 of this were associated with subjects with SMI). 13% of the windows were associated with all awake, 18% of the windows were associated with in transition, and 69% of the windows were associated with all asleep. Of those in transition for a binary classification omitting windows associated with in transition, 17% of windows were associated with half or less asleep and 83% of windows were associated with more than half asleep. Thus, the sleep fraction histogram 300 shows bias toward sleep data, making waking periods harder to detect.

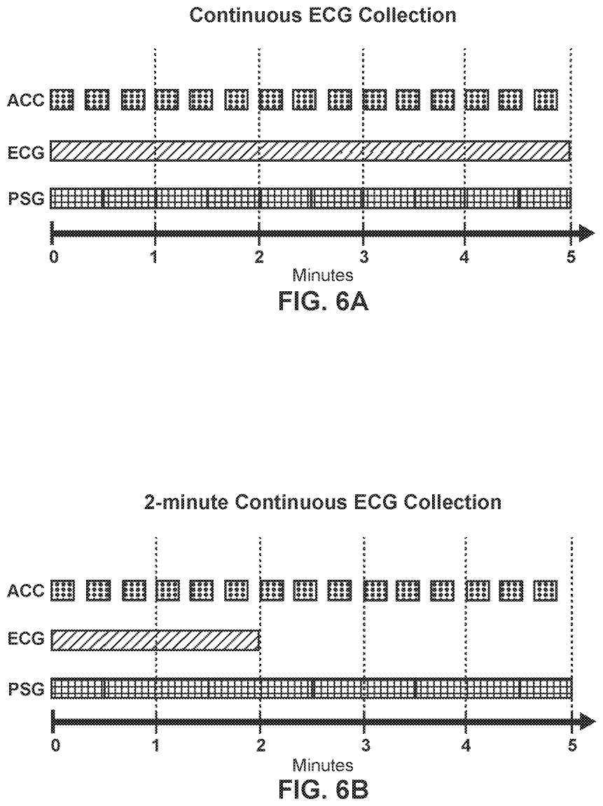

FIG. 4A illustrates an example of a sampling rate of accelerometer and ECG data in a time series with PSG labeling data. The accelerometer data in FIGS. 4A-4C and 6A-6B is illustrated as “ACC.” In the example of FIG. 4A, the sampling rate of the accelerometer and ECG sensors are each three blocks per minute, or fifteen blocks per five-minute window. A block may refer to a set of data for which a sensor generated data. For example, in a given one minute increment, a sensor 112 may generate or transmit sensor data in a 14-second or other interval of time. The 14-second or other interval of time for which the sensor 112 generated or transmitted the sensor data may be referred to as a “block.” It should be noted that other sampling rates may be used as well. The PSG labeling data (illustrated in the figures as “PSG”) may be included at a rate of every 30 seconds, the subject's sleep state is known from the PSG test. The ACC and ECG data will be used to generate a feature set, which is correlated with the known sleep state from the PSG test.

FIG. 4B illustrates an example of down-sampled accelerometer data and maintained sampling rate of ECG data in a time series with PSG labeling data. In this example, the sampling rate of the accelerometer is down-sampled to two ACC blocks per minute, or ten ACC blocks per five-minute window, while the sampling rate of the ECG sensor is three ECG blocks per minute and the PSG labeling data is provided every 30 seconds.

FIG. 4C illustrates another example of down-sampled accelerometer data and maintained sampling rate of ECG data in a time series with PSG labeling data. In this example, the sampling rate of the accelerometer is down-sampled to one ACC block per minute, or five ACC blocks per five-minute window, while the sampling rate of the ECG sensor is three ECG blocks per minute and the PSG labeling data is provided every 30 seconds.

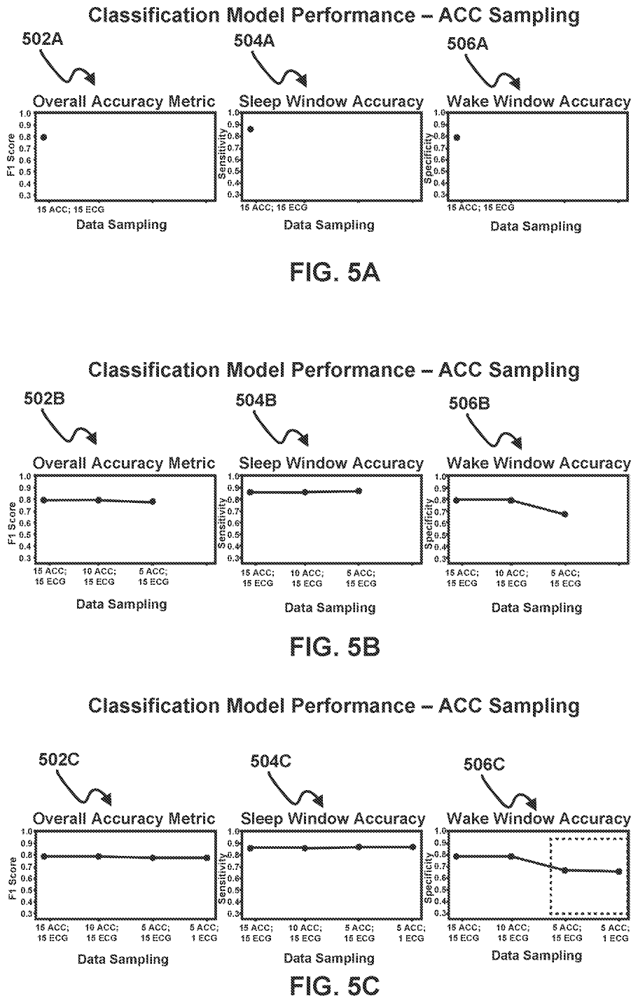

FIG. 5A illustrates plots 502A, 504A, 506A of model performance at a sampling rate of accelerometer and ECG data in a time series with PSG labeling data shown in FIG. 4A. Plot 502A shows an overall accuracy metric (f1 score) of predictions when the machine-learning model 168 is trained with the sampling rates shown in FIG. 4A. The overall accuracy metric may be an aggregate (such as mean) of sleep window accuracy and wake window accuracy. Plot 502B shows the sensitivity of the machine-learning model 168 trained with the sampling rates shown in FIG. 4A. Sleep window accuracy may refer to how accurately the machine-learning model 168 predicted that a subject was asleep during a given window. Plot 502C shows the specificity of the machine-learning model 168 trained with the sampling rates shown in FIG. 4A. Wake window accuracy may refer to how accurately the machine-learning model 168 predicted that a subject was awake during a given window.

FIG. 5B illustrates plots 502B, 504B, 506B showing the effects of varying the sampling rate of ACC data while maintaining the sampling rate of the ECG data. The x-axis shows different combinations of sampling rates shown in FIGS. 4A-C. Model performance on wake windows begins to degrade significantly with five ACC blocks per window.

FIG. 5C illustrates plots 502C, 504C, 506C showing the effects of varying the sampling rate of ACC data while maintaining the sampling rate of the ECG data and also showing a plot point of reduced ECG data sampling rate.

The x-axis shows different combinations of sampling rates shown in FIGS. 4A-C and also down-sampled ECG data at one block per window combined with five ACC blocks per window. Model performance on wake windows begins to degrade significantly with five ACC blocks per window. Fifteen ECG blocks does not provide significantly better performance than one ECG Block, indicating that the mean heart rate is influencing the model but the heart rate variability metrics may not be influencing the model.

FIG. 6A illustrates an example of a sampling rate of accelerometer and continuous ECG data in a time series with PSG labeling data. As shown, a sampling rate of fifteen blocks of ACC data per window is used with a continuous rate for the ECG data throughout a five-minute window.

FIG. 6B illustrates an example of a sampling rate of accelerometer data and continuous ECG data for a portion of a window in a time series with PSG labeling data. As shown, a sampling rate of fifteen blocks of ACC data per window is used with a continuous rate for the ECG data for only a portion of a five-minute window (such as the first two minutes of the five minute window) after which sampling is discontinued for the remainder of the window. In both FIGS. 6A and 6B, the PSG data is continuous.

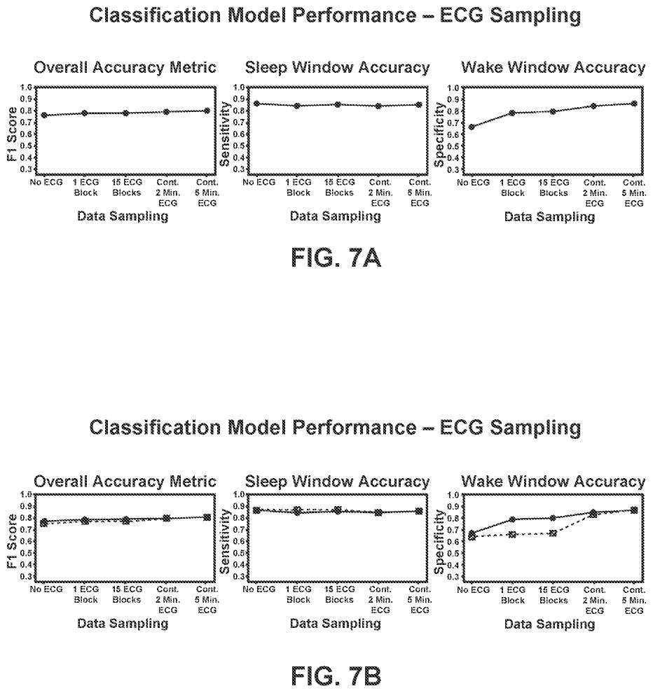

FIG. 7A illustrates plots 702A, 704A, and 706A showing the effects of varying the sampling rate of ECG data while maintaining the sampling rate of the ACC data. Continuous ECG data provides better wake window classification than block sampling.

FIG. 7B illustrates plots 702B, 704B, and 706B showing the effects of varying the sampling rate of ECG data while maintaining the sampling rate of the ACC data at fifteen ACC blocks per window overlaid with the effects of varying the sampling rate of ECG data while maintaining the sampling rate of the ACC data at five ACC blocks per window. The performance gap between continuous and block-sampled ECG is greater with 5 ACC Blocks than 15 ACC Blocks.

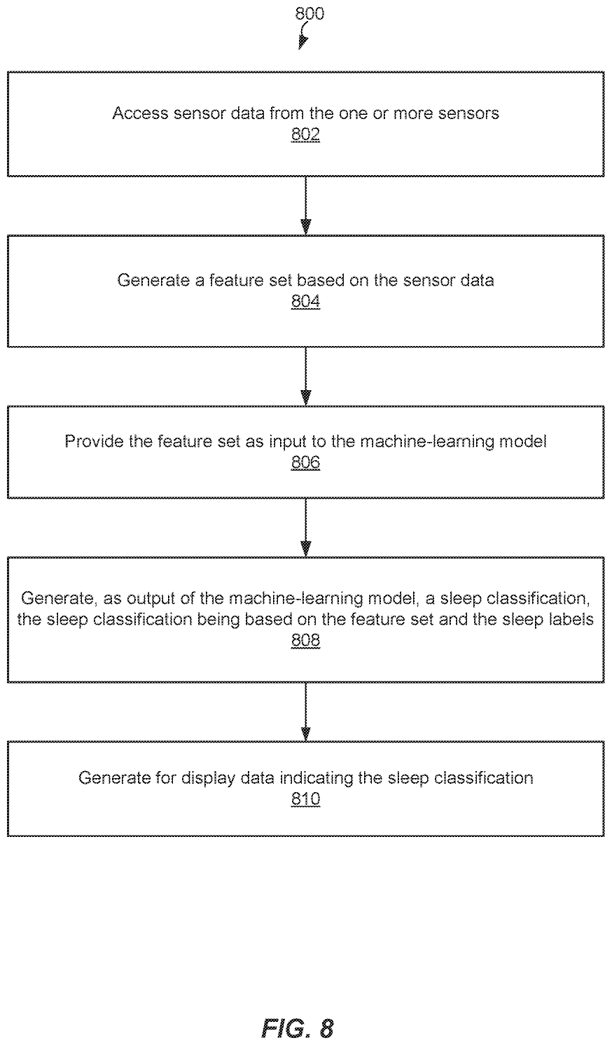

FIG. 8 illustrates an example of a method 800 of generating physiological state classifications based on the machine-learning model. At 802, the method 800 may include accessing sensor data from one or more sensors (such as one or more sensors 122A-N). The sensor data may include, for example, measurements of a subject from any single or combination of one or more of an accelerometer, an ECG sensor, and/or other sensor. At 804, the method 800 may include generating a feature set based on the sensor data. An example of the feature set may include the feature set 211 or the filtered feature set 213 illustrated in FIG. 2. At 806, the method 800 may include providing the feature set as input to a machine-learning model. An example of the machine-learning model may include the machine-learning model 168. At 808, the method 800 may include generating, as output of the machine-learning model, a sleep classification for the window, the sleep classification being based on the feature set and the sleep labels. At 810, the method 800 may include generating for display data indicating the sleep classification.

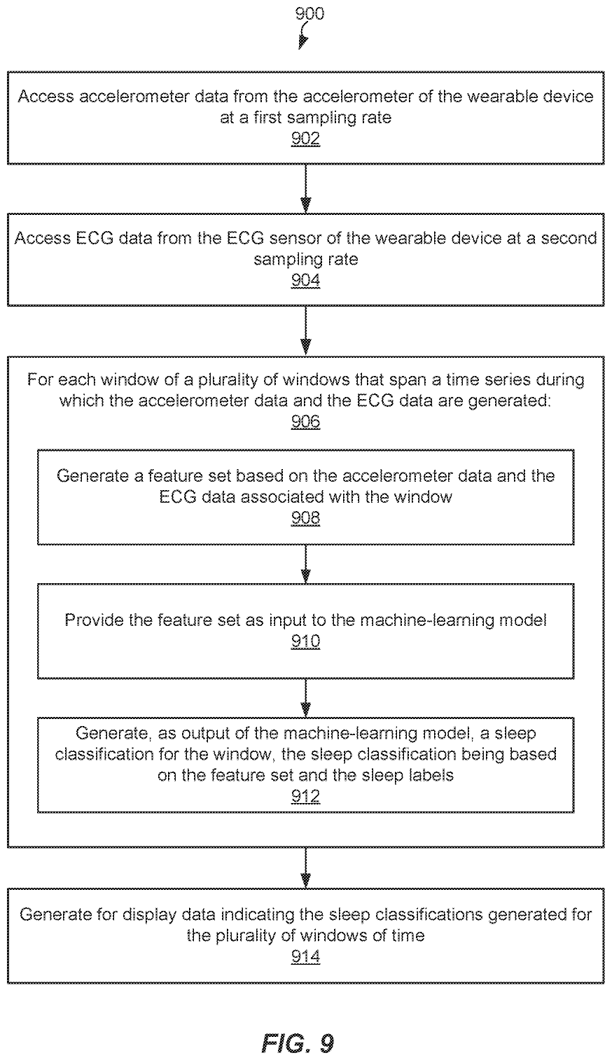

FIG. 9 illustrates an example of a method 900 of generating sleep classifications based on the machine-learning model. At 902, the method 900 may include accessing accelerometer data from the accelerometer (such as a sensor 112A) of a wearable device (such as a sensor device 110 configurated as a wearable device) at a first sampling rate. FIGS. 4A-4C and FIGS. 6A-6B shows examples of the first sampling rate of the accelerometer data. Other sampling rates may be used as well.

At 904, the method 900 may include accessing ECG data from an ECG sensor (such as a sensor 112B) of the wearable device at a second sampling rate. FIGS. 4A-4C and FIGS. 6A-6B shows examples of the second sampling rate of the ECG data. Other sampling rates may be used as well.

At 906, the method 900 may include, processing the accelerometer data and the ECG data based on windows (such as windows W1-W(N) illustrated in FIG. 2). For each window of a plurality of windows that span a time series during which the accelerometer data and the ECG data are generated, the method 900 may include processing at 908, 910, and 912.

At 908, the method 900 may include generating a feature set based on the accelerometer data and the ECG data associated with the window. An example of the feature set may include the feature set 211 or the filtered feature set 213 illustrated in FIG. 2.

At 910, the method 900 may include providing the feature set as input to a machine-learning model. An example of the machine-learning model may include the machine-learning model 168, which may be trained based on the feature set and sleep labels. The feature set may be derived from training accelerometer data from the accelerometer and training ECG data from the ECG sensor. Each of the sleep labels may indicate a sleep stage during a window of time determined from a sleep study conducted while collecting the training accelerometer data and the training ECG data to train the machine-learning model. The training accelerometer data and the training ECG data respectively refers to accelerometer data and ECG data collected during a training phase of the machine-learning model, such as during training by the model training system 160.

At 912, the method 900 may include generating, as output of the machine-learning model, a sleep classification for the window, the sleep classification being based on the feature set and the sleep labels. The sleep classification may be a binary classification, a one-class classification, or a multi-class classification.

At 914, the method 900 may include generating for display data indicating the sleep classifications generated for the plurality of windows of time. For example, the method 900 may include generating a sleep profile that shows windows of sleep predicted for the measured time period.

A subject refers to an animal, such as a mammalian species (preferably human) or avian (e.g., bird) species, or other organism for which a physiological state such as a sleep stage may be determined. More specifically, a subject can be a vertebrate, e.g., a mammal such as a mouse, a primate, a simian or a human. Animals include farm animals, sport animals, and pets. A subject can be a healthy individual, an individual that has symptoms or signs or is suspected of having a disease (including physical or mental disease) or a predisposition to the disease, or an individual that is in need of therapy or suspected of needing therapy.

### Examples of Systems and Computing Devices

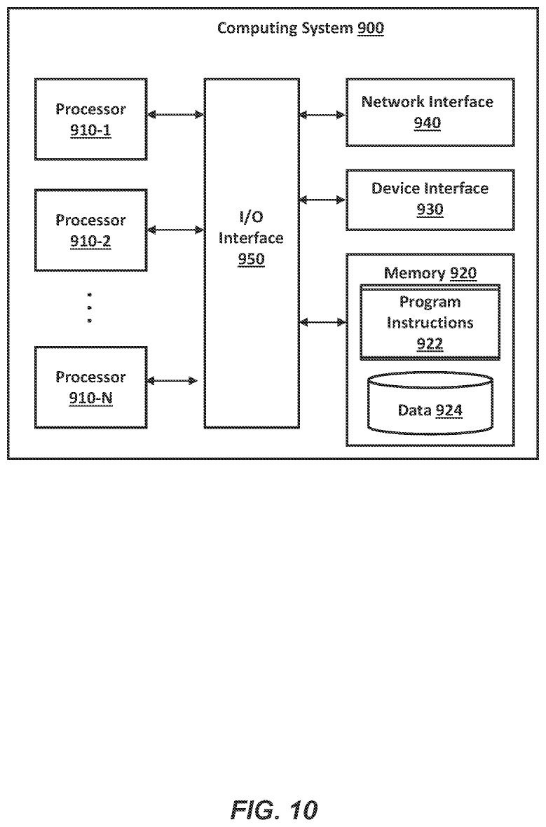

FIG. 10 illustrates an example of a computing system implemented by one or more of the features illustrated in FIG. 1. Various portions of systems and methods described herein, may include or be executed on one or more computer systems similar to computing system 1000. Further, processes and modules described herein may be executed by one or more processing systems similar to that of computing system 1000. In some embodiments, sensor device 110, computing device 120, computing device 140, or other components of system 100 may include some or all of the components and features of computing system 1000.

Computing system 1000 may include one or more processors (for example, processors 1010-1-1010-N) coupled to system memory 1020, an input/output I/O device interface 1030, and a network interface 1040 via an input/output (I/O) interface 1050. A processor may include a single processor or a plurality of processors (for example, distributed processors). A processor may be any suitable processor capable of executing or otherwise performing instructions. A processor may include a central processing unit (CPU) that carries out program instructions to perform the arithmetical, logical, and input/output operations of computing system 1000. A processor may execute code (for example, processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (for example, system memory 1020). Computing system 1000 may be a uni-processor system including one processor (for example, processor 1010-1), or a multi-processor system including any number of suitable processors (for example, 1010-1-1010-N). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus may also be implemented as, special purpose logic circuitry, for example, an FPGA (field programmable gate array) or an ASIC (application specific integrated circuit). Computing system 1000 may include a plurality of computing devices (for example, distributed computer systems) to implement various processing functions.

I/O device interface 1030 may provide an interface for connection of one or more I/O devices to computing system 1000. I/O devices may include devices that receive input (for example, from a user) or output information (for example, to a user). I/O devices may include, for example, graphical user interface presented on displays (for example, a cathode ray tube (CRT) or liquid crystal display (LCD) monitor), pointing devices (for example, a computer mouse or trackball), keyboards, keypads, touchpads, scanning devices, voice recognition devices, gesture recognition devices, printers, audio speakers, microphones, cameras, or the like. I/O devices may be connected to computing system 1000 through a wired or wireless connection. I/O devices may be connected to computing system 1000 from a remote location. I/O devices located on remote computer system, for example, may be connected to computing system 1000 via network interface 1040.

Network interface 1040 may include a network adapter that provides for connection of computing system 1000 to a network. Network interface 1040 may facilitate data exchange between computing system 1000 and other devices connected to the network. Network interface 1040 may support wired or wireless communication. The network may include an electronic communication network, such as the Internet, a local area network (LAN), a wide area network (WAN), a cellular communications network, or the like.

System memory 1020 may store program instructions 1022 or data 1024. Program instructions 1022 may be executable by a processor (for example, one or more of processors 1010-1-1010-N) to implement one or more embodiments of the present techniques. Program instructions 1022 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (for example, one or more scripts stored in a markup language document), in a single file dedicated to the program in question, or in multiple coordinated files (for example, files that store one or more modules, sub programs, or portions of code). A computer program may be deployed to be executed on one or more computer processors located locally at one site or distributed across multiple remote sites and interconnected by a communication network.

System memory 1020 may include a tangible program carrier having program instructions stored thereon. A tangible program carrier may include a non-transitory computer readable storage medium. A non-transitory computer readable storage medium may include a machine-readable storage device, a machine-readable storage substrate, a memory device, or any combination thereof. Non-transitory computer readable storage medium may include non-volatile memory (for example, flash memory, ROM, PROM, EPROM, EEPROM memory), volatile memory (for example, random access memory (RAM), static random access memory (SRAM), synchronous dynamic RAM (SDRAM)), bulk storage memory (for example, CD-ROM and/or DVD-ROM, hard-drives), or the like. System memory 1020 may include a non-transitory computer readable storage medium that may have program instructions stored thereon that are executable by a computer processor (for example, one or more of processors 1010-1-1010-N) to cause the subject matter and the functional operations described herein. A memory (for example, system memory 1020) may include a single memory device and/or a plurality of memory devices (for example, distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times.

I/O interface 1050 may coordinate I/O traffic between processors 1010-1-1010-N, system memory 1020, network interface 1040, I/O devices, and/or other peripheral devices. I/O interface 1050 may perform protocol, timing, or other data transformations to convert data signals from one component (for example, system memory 1020) into a format suitable for use by another component (for example, processor 1010-1, processor 1010-2, . . . , processor 1010-N). I/O interface 1050 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computing system 1000 or multiple computing systems 1000 configured to host different portions or instances of embodiments. Multiple computing systems 1000 may provide for parallel or sequential processing/execution of one or more portions of the techniques described herein.

Those skilled in the art will appreciate that computing system 1000 is merely illustrative and is not intended to limit the scope of the techniques described herein. Computing system 1000 may include any combination of devices or software that may perform or otherwise provide for the performance of the techniques described herein. For example, computing system 1000 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computing system 1000 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (for example, as instructions or structured data) on a computer-accessible medium or a portable article to be read by an appropriate drive, various examples of which are described above. In some embodiments, instructions stored on a computer-accessible medium separate from computing system 1000 may be transmitted to computing system 1000 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (for example, content) is said to be supplied or otherwise provided, the information may be provided by sending instructions to retrieve that information from a content delivery network.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein, but it should be understood that the techniques are independently useful, and some embodiments address only a subset of such problems or offer other, unmentioned benefits that will be apparent to those of skill in the art reviewing the present disclosure. Due to cost constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (in other words, meaning having the potential to), rather than the mandatory sense (in other words, meaning must). The words “include”, “including”, and “includes” and the like mean including, but not limited to. As used throughout this application, the singular forms “a,” “an,” and “the” include plural referents unless the content explicitly indicates otherwise. Thus, for example, reference to “an element” or “a element” includes a combination of two or more elements, notwithstanding use of other terms and phrases for one or more elements, such as “one or more.” The term “or” is, unless indicated otherwise, non-exclusive, in other words, encompassing both “and” and “or.” Terms describing conditional relationships, for example, “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, for example, “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, for example, the antecedent is relevant to the likelihood of the consequent occurring. Statements in which a plurality of attributes or functions are mapped to a plurality of objects (for example, one or more processors performing steps A, B, C, and D) encompasses both all such attributes or functions being mapped to all such objects and subsets of the attributes or functions being mapped to subsets of the attributes or functions (for example, both all processors each performing steps A-D, and a case in which processor 1 performs step A, processor 2 performs step B and part of step C, and processor 3 performs part of step C and step D), unless otherwise indicated. Similarly, reference to “a computer system” performing step A and “the computer system” performing step B may include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, in other words, each does not necessarily mean each and every. Limitations as to sequence of recited steps should not be read into the claims unless explicitly specified, for example, with explicit language like “after performing X, performing Y,” in contrast to statements that might be improperly argued to imply sequence limitations, like “performing X on items, performing Y on the X′ ed items,” used for purposes of making claims more readable rather than specifying sequence. Statements referring to “at least Z of A, B, and C,” and the like (for example, “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. Features described with reference to geometric constructs, like “parallel,” “perpendicular/orthogonal,” “square”, “cylindrical,” and the like, should be construed as encompassing items that substantially embody the properties of the geometric construct, for example, reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first”, “second”, “third,” “given” and so on, if used in the claims, are used to distinguish or otherwise identify, and not to show a sequential or numerical limitation. As is the case in ordinary usage in the field, data structures and formats described with reference to uses salient to a human need not be presented in a human-intelligible format to constitute the described data structure or format, for example, text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer implemented instructions, commands, and the like are not limited to executable code and may be implemented in the form of data that causes functionality to be invoked, for example, in the form of arguments of a function or API call. To the extent bespoke noun phrases are used in the claims and lack a self-evident construction, the definition of such phrases may be recited in the claim itself, in which case, the use of such bespoke noun phrases should not be taken as invitation to impart additional limitations by looking to the specification or extrinsic evidence.

In this patent, to the extent any U.S. patents, U.S. patent applications, or other materials (for example, articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

This written description uses examples to disclose the embodiments, including the best mode, and to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system, comprising:
a wearable device comprising an accelerometer that senses movement of a subject that wears the wearable device and an electrocardiogram (ECG) sensor that senses a heartrate of the subject;
a computing device, comprising:
a memory configured to store a machine-learning model that generates a sleep classification based on input sensor data, wherein the machine-learning model is trained based on a feature set and sleep labels, the feature set being derived from training accelerometer data from the accelerometer and training ECG data from the ECG sensor and the sleep labels each indicating a sleep stage during a window of time determined from a sleep study conducted while collecting the training accelerometer data and the training ECG data to train the machine-learning model;
a processor programmed to:
access accelerometer data from the accelerometer of the wearable device at a first sampling rate;
access ECG data from the ECG sensor of the wearable device at a second sampling rate;
for each window of a plurality of windows that span a time series during which the accelerometer data and the ECG data are generated:
generate a feature set based on the accelerometer data and the ECG data associated with the window;
provide the feature set as input to the machine-learning model;
generate, as output of the machine-learning model, a sleep classification for the window, the sleep classification being based on the feature set and the sleep labels; and
generate for display data indicating the sleep classifications generated for the plurality of windows of time.

2. The system of claim 1, wherein the first sampling rate of the accelerometer data is higher than the second sampling rate of the ECG data.

3. The system of claim 1, wherein the first sampling rate of the accelerometer data is equal to the second sampling rate of the ECG data.

4. The system of claim 1, wherein the second sampling rate of the ECG data is continuous.

5. The system of claim 1, wherein the wearable device is further programmed to adaptively up-sample the ECG data.

6. The system of claim 5, wherein to adaptively up-sample the ECG data, the wearable device is further programmed to up-sample the ECG data from a non-continuous sampling rate to a continuous sampling rate.

7. The system of claim 5, wherein to adaptively up-sample the ECG data, the wearable device is further programmed to up-sample the ECG data from a non-continuous sampling rate to a continuous sampling rate throughout a given window.

8. The system of claim 5, wherein to adaptively up-sample the ECG data, the wearable device is further programmed to up-sample the ECG data from a non-continuous sampling rate to a continuous sampling rate for only a portion of a given window.

9. The system of claim 5, wherein to adaptively up-sample the ECG data, the wearable device is further programmed to:
determine a triggering event has occurred, wherein the ECG data is up-sampled responsive to the triggering event.

10. The system of claim 9, wherein the triggering event comprises detection of reduced activity by the subject below a threshold value as indicated by the accelerometer data.

11. The system of claim 1, wherein the machine-learning model comprises:
a conditional random field model.

12. The system of claim 1, wherein the machine-learning model comprises: a long-term short-term memory.

13. The system of claim 1, wherein the machine-learning model comprises: a light gradient boosting machine

14. The system of claim 1, wherein to generate the feature set, the computing system is further programmed to:
generate, specifically for the subject, a baseline for a feature in the feature set derived from the accelerometer data and/or the ECG data, the baseline being used to customize the sleep classification for the subject.

15. The system of claim 1, wherein to generate the feature set, the computing system is further programmed to:
for a feature of the feature set in each window:
generate a first average value for the window for each of the accelerometer data and/or the ECG data;
generate a second average value for a prior window for each of the accelerometer data and/or the ECG data;
determine a difference between the second average value and the first average value; and
use the difference in the feature set for the feature.

16. The system of claim 1, wherein the feature set comprises a filtered feature set that was selected during feature selection.

17. A method, comprising:
accessing, by a computing device, accelerometer data from an accelerometer of a wearable device at a first sampling rate;
accessing, by the computing device, electrocardiogram (ECG) data from an ECG sensor of the wearable device at a second sampling rate;
for each window of a plurality of windows that span a time series during which the accelerometer data and the ECG data are generated:
generating, by the computing device, a feature set based on the accelerometer data and the ECG data associated with the window;
providing, by the computing device, the feature set as input to a machine-learning model, wherein the machine-learning model is trained based on a feature set and sleep labels, the feature set being derived from training accelerometer data from the accelerometer and training ECG data from the ECG sensor and the sleep labels each indicating a sleep stage during a window of time determined from a sleep study conducted while collecting the training accelerometer data and the training ECG data to train the machine-learning model;
generating, by the computing device, as output of the machine-learning model, a sleep classification for the window, the sleep classification being based on the feature set and the sleep labels; and
generating, by the computing device, for display data indicating the sleep classifications generated for the plurality of windows of time.

18. A non-transitory computer-readable medium that stores instructions that, when executed by a processor programs the processor to:
access accelerometer data from an accelerometer of a wearable device at a first sampling rate;
access electrocardiogram (ECG) data from an ECG sensor of the wearable device at a second sampling rate;
for each window of a plurality of windows that span a time series during which the accelerometer data and the ECG data are generated:
generate a feature set based on the accelerometer data and the ECG data associated with the window;
provide the feature set as input to a machine-learning model, wherein the machine-learning model is trained based on a feature set and sleep labels, the feature set being derived from training accelerometer data from the accelerometer and training ECG data from the ECG sensor and the sleep labels each indicating a sleep stage during a window of time determined from a sleep study conducted while collecting the training accelerometer data and the training ECG data to train the machine-learning model;
generate, as output of the machine-learning model, a sleep classification for the window, the sleep classification being based on the feature set and the sleep labels; and
generate for display data indicating the sleep classifications generated for the plurality of windows of time.

19. A sensor device, comprising:
a first sensor configured to measure a subject at a first sampling rate;
a second sensor configured to measure the subject at a second sampling rate; and
a processor programmed to:
detect a triggering event based on sensor data from the first sensor;
adjust the second sampling rate based on the triggering event.

20. A method, comprising:
measuring, by a first sensor of a sensor device, a subject at a first sampling rate;
measuring, by a second sensor of the sensor device, the subject at a second sampling rate; and
detecting, by the sensor device, a triggering event based on sensor data from the first sensor;
adjusting, by the sensor device, the second sampling rate based on the triggering event.

21. A computing device, comprising:
a memory configured to store a machine-learning model that generates a sleep classification based in input sensor data, wherein the machine-learning model is trained based on a feature set and sleep labels, the feature set being derived from training sensor data from one or more sensors and the sleep labels each indicating a sleep stage as determined from a sleep study conducted while collecting the training sensor data to train the machine-learning model;
a processor programmed to:
access sensor data from the one or more sensors;
generate a feature set based on the sensor data;
provide the feature set as input to the machine-learning model;
generate, as output of the machine-learning model, a sleep classification, the sleep classification being based on the feature set and the sleep labels; and
generate for display data indicating the sleep classification.

22. A method, comprising:
accessing, by a computing device, sensor data from one or more sensors;
generating, by the computing device, a feature set based on the sensor data;
providing, by the computing device, the feature set as input to a machine-learning model;
generating, by the computing device, as output of the machine-learning model, a sleep classification, the sleep classification being based on the feature set and sleep labels; and
generating, by the computing device, for display data indicating the sleep classification.

23. A non-transitory computer-readable medium that stores instructions that, when executed by a processor programs the processor to:
access sensor data from one or more sensors;
generate a feature set based on the sensor data;
provide the feature set as input to a machine-learning model;
generate, as output of the machine-learning model, a sleep classification, the sleep classification being based on the feature set and sleep labels; and
generate for display data indicating the sleep classification.
